(19) United States
(12) Patent Application Publication
(10) Pub. No.: US 2004/0098816 A1
(43) Pub. Date: May 27, 2004

(54) Method and kit for the gradual permanent coloring of hair

(75) Inventors: Van Au, New City, NY (US); Stephen Alan Madison, New City, NY (US); John Brian Bartolone, Bridgeport, CT (US)

Correspondence Address: UNILEVER, PATENT DEPARTMENT, 45 RIVER ROAD, EDGEWATER, NJ, 07020, US

(73) Assignee: Unilever Home & Personal Care USA Division of Conopco, Inc.

(21) Appl. No.: 10/674,261

(22) Filed: Sep. 29, 2003

Related U.S. Application Data
(63) Continuation-in-part of application No. 10/306,605, filed on Nov. 27, 2002.

(51) Int. Cl. A61K 7/13

(52) U.S. Cl. 8/405

(57) ABSTRACT

A method and kit for permanently dyeing hair is provided which includes subjecting said hair to a number of treatments having a set time interval between each two consecutive such treatments. The method and kit utilize a colorant composition with a part A and part B. The overall composition will contain an ammonium carbonate or carbamate salt in combination with a chelant, the latter being present at levels of at least 1%. The combination of ammonium salts and chelant achieve reduced hair damage, enhanced color deposition and enhanced bleaching. Particularly preferred is a combination of ammonium carbonate and ethylenediaminetetraacetic acid salt.

### CROSS REFERENCES

This application is a Continuation-In-Part of Ser. No. 10/306,605, filed Nov. 27, 2002.

### BACKGROUND OF THE INVENTION

The present invention relates to a method and kit for the permanent coloring of hair with longer lasting conditioning and with minimized hair damage.

Most hair coloring products fall under three major groupings:

1. Temporary hair color

2. Semi permanent hair color

3. Permanent hair color

Temporary color is a leave on product that causes minimal damage to the hair. However, temporary color causes stains, and leaches out under rain or with perspiration. Temporary color washes out with the next shampoo. Temporary color also does not give any control to the consumer over the amount of color deposited or the permanency of the color supplied. Temporary color does not result in a wide variety of colors and it has only a limited appeal.

Semi-permanent hair color comes as a rinse, and it causes minimal damage to the hair. However, semi-permanent hair color washes out to some degree with each shampoo and washes out completely within about 4 to 6 shampoos. Semi permanent hair color does not give the consumer any control regarding the amount of color deposited or the permanency of the color. Semi-permanent hair color has limited popularity with consumers.

Permanent hair color generally comes in two parts: a dye solution and a developer solution. Because of the damaging nature of current permanent dye treatments, most home coloring products come with a post treatment conditioner. In a permanent hair coloring treatment, the dye solution and the developer solution are mixed and then applied to the hair, which is then left for about 25 to about 35 minutes. The hair is then rinsed with water, treated with a post treatment conditioner, and then rinsed again with water.

The application of the dye solution and the developer solution affords permanent hair coloring. However, this method does not provide any conditioning benefit. The conditioning benefit comes through application of the post treatment conditioner, and it is only temporary. The conditioning benefit is lost with the next shampoo. Moreover, with permanent hair coloring treatments, shampooing the hair is usually not recommended after said treatments. Thus, hair is left feeling dirty, and can stain towels and pillows.

Coloring products need to be applied every four to six weeks since hair grows out of the scalp at the rate of approximately one half inch per month. Each coloring application causes damage to the hair, and that damage is cumulative. Hair touch ups after the initial treatment would also damage hair more.

It would be desirable to develop a method and kit with one or more benefits such as permanently and effectively coloring hair that also conditions, gives hair a soft clean feel, and minimizes the damage caused to hair by the coloring process. The present invention provides such a method and kit.

Patents related to the field of this invention are as follows:

U.S. Pat. No. 5,131,912 discloses durable two-part hair dyeing agents composed of a first agent comprising as essential components at least one compound that forms HCO₃— by dissociation in water, an alkali generating substantially no irritating odor and a dye for hair and having a pH of 8.2 to 9.0. The second agent comprises as essential components hydrogen peroxide and a buffer solution and having a pH of 2.0 to 4.0. The weight ratio of the first agent and the second agent to be mixed being such that the pH of the mixture of the two is in a range of from 6.5 to 7.9. These two-part hair dyeing agents require only a short dyeing time, create little damage to hair and no irritating or disagreeable odor and have high dyeing effect.

U.S. Pat. No. 5,525,123, discloses a hair dyeing composition based on oxidation dyestuff precursors which dye and brighten the hair. Besides at least one developing and at least one coupling agent, the composition contains at least one metal salt and at least one ammonium compound selected from ammonium chloride, ammonium sulfate, ammonium carbonate, ammonium bicarbonate, and ammonium carbamate, having a pH-value between 8 and 11, preferably from 9 to 10, after admixture with an oxidizing agent in the ready-to-use preparation.

U.S. Patent Appln. No. 2003/0051297 A1 discloses a method for permanently dyeing hair which comprises subjecting the hair to a number of treatments, having a set time interval between each two consecutive such treatments.

U.S. Pat. No. 4,104,021 discloses a process in which human hair is dyed in successive treatments at selected intervals with oxidation colors (aromatic primary amines and amino phenols) admixed in each treatment with an oxidizing agent (H₂O₂ or a derivative thereof. The quantity of oxidation colorant applied in each treatment is substantially the same and the quantity of oxidizing agent is increased from the first to the last treatment to effect a gradual increase in depth of shade. The mixture is allowed to remain on the hair for substantially the same time in each treatment, followed by removal by rinsing.

U.S. Pat. No. 4,529,404 discloses an autoxidizable hair dye preparation capable of coloring or darkening hair when applied thereto and exposed to the atmosphere comprising a mixture of (I) at least one p-phenylene diamine compound, or an acid addition salt thereof, and (II) at least one 1,2,4-benzenetriol compound, each compound optionally containing nuclearly substituted C₁₋₄alkyl, alkoxy, hydroxyalkyl or halogen. The preparation is preferably applied and exposed to the atmosphere repeatedly until the desired degree of darkening or color build-up is attained.

The preparations of this invention may also contain known additives or assistants such as hair grooming agents, for example quaternized vinyl pyrrolidone copolymers, carboxyvinyl polymers and the like, plasticizers, conditioners, thickeners, slip and wetting agents such as polyoxyethylenated fatty (e.g. lauryl) alcohols, stearyldimethylammonium chloride, silicone copolymer, foam boosters, preservatives, perfumes and the like.

U.S. Pat. No. 5,968,486 describes a shampoo composition for lightening and highlighting hair which comprises a peroxygen compound and an anionic sulfonate, the composition having a pH less than 5. There is also described an invention directed to a method for lightening and highlighting hair which comprises shampooing the hair with a lightening and highlighting effective amount of a composition of the invention.

### SUMMARY OF THE INVENTION

The present invention relates to a method for achieving permanent desired hair color change through the use of daily hair care compositions. The daily hair care compositions comprise a mixture of two compositions, part A and part B. These are provided to a consumer in a kit with instructions for coloring the hair.

Accordingly, a method for permanently dyeing hair is provided which comprises subjecting a person's hair to a number of treatments, having a set time interval between each two consecutive treatments, wherein each treatment comprises:

a) contacting the hair with a recently prepared mixture of a colorant composition comprising:

A) an alkaline dye composition comprising:

(i) an effective amount to color hair of at least one dye intermediate;

(ii) from about 0.1 to about 25% by weight based on the colorant composition of a water soluble ammonium carbonate or carbamate salt;

(iii) from 1 to 5% by weight based on the colorant composition of a chelant; and

(iv) a cosmetically acceptable carrier;

B) an oxidizing composition comprising:

(i) from 0.1 to 15% by weight based on the coloring composition of a peroxide compound; and

(ii) a cosmetically acceptable carrier;

b) rinsing the mixture from the hair with water.

Further, a kit for permanently dyeing hair is provided which comprises:

a) a colorant composition packaged as a part A and a part B separate from one another until just before use,

part A being an alkaline dye composition comprising:

(i) an effective amount to color hair of at least one dye intermediate;

(ii) from about 0.1 to about 25% by weight of the colorant composition of a water soluble ammonium carbonate or carbamate salt;

(iii) from 1 to 5% by weight based on the colorant composition of a chelant; and

(iv) a cosmetically acceptable carrier.

part B being an oxidizing composition comprising:

(i) from 0.1 to 15% by weight based on the colorant composition of a peroxide compound; and

(ii) a cosmetically acceptable carrier.

b) instructions for use of the colorant composition comprising applying a recently made mixture of part A and part B onto a person's hair followed by a rinse with water.

Advantageously the method and instructions for the kit of the present invention may advise that each treatment be from 2 to about 30 in number, but preferably at least 6 in number, the set time interval between each of two consecutive treatments is between about 8 hours and 30 days, and contact time of the mixture on the hair is for a period of about 5 seconds to about 5 minutes, preferably only up to 3 minutes per treatment.

### DETAILED DESCRIPTION OF THE INVENTION

As used here % means weight % of the total composition (part A and B) unless 10 otherwise specified. The term “recently” means within a very short interval of time such as within a few seconds or minutes, such as within 0.01 seconds to 120 seconds, or within 0.1 seconds to 60 seconds, or within 0.5 second to within 30 second or within 2 seconds to within 20 seconds.

Now it has been found that excellent permanent coloring can be achieved while substantially avoiding the normally occurring damage with previously known systems. Damage control is achieved through addition of a relatively high level of chelant in combination with a water soluble ammonium carbonate or carbamate salt. The chelant may be incorporated into either part A of the colorant system which part delivers the dye intermediate or formulated into part B containing a peroxide compound. The water soluble ammonium carbonate or carbamate salt is formulated into part A, the latter being an alkaline composition.

The term “chelant” (or “chelating agent” or “sequestering agent”) is well known in the art and refers to a molecule or a mixture of different molecules each capable of forming a chelate with a metal ion. A chelate is an inorganic complex in which a compound (chelant) is coordinated to a metal ion at two or more points so that there is a ring of atoms including the metals. Chelants contain two or more electron donor atoms that form coordination bonds with the metal ion.

When used herein, the term “chelant” includes all salts and derivatives comprising the same functional structure as the parent chelant they are referring to and have similar or better chelating properties. These terms include alkali metal, alkaline earth, ammonium, substituted ammonium salts (e.g. monoethanolammonium, diethanolammonium, triethanolammonium), esters of chelants having an acidic moiety and mixtures thereof, in particular all sodium, potassium or ammonium salts. The term “Derivatives” also includes “chelating surfactant” compounds (these are chelants modified to bear a surfactant moiety while keeping the same chelating functionality, see U.S. Pat. No. 5,284,972, “N-acyl-N,N′,N′-ethylenediaminetriacetic acid” for an example of modified ethylenediaminetriacetic acid). The term “Derivatives” also includes large molecules comprising one or more chelating groups having the same functional structure as the parent chelants. An example of these large molecules is polymeric EDDS (ethylenediaminedisuccinic acid).

Preferred chelants for use herein are carboxylic acids (in partricular aminocarboxylic acids), phosphonic acids (in particular aminophosphonic acids), and polyphosphoric acids (in particular linear polyphosphoric acids), their salts and derivatives.

### Aminocarboxylic Acid Chelants

Carboxylic acid chelants as defined herein are chelants having at least one carboxylic acid moiety (—COOH).

Examples of aminocarboxylic acid chelants suitable for use herein include nitrilotriacetic acid and polyaminocarboxylic acids such as diethylenetriamine pentaacetic acid (DTPA), ethylenediamine disuccinic acid (EDDS), ethylenediamine diglutaric acid (EDGA), 2-hydroxypropylenediamine disuccinic acid (HPDS), glycinamide-N,N′-disuccinic acid (GADS), ethylenediamine-N-N′-diglutaric acid (EDDG), 2-hydroxypropylenediamine-N-N′-disuccinic acid (HPDDS), ethylenediaminetetraacetic acid (EDTA), dipicolinic acid (DPA), salts thereof and derivatives thereof.

Other suitable aminocarboxylic chelants for use herein are iminodiacetic acid derivatives such as N-2-hydroxyethyl N,N diacetic acid or glyceryl imino diacetic acid (described in EP-A-317,542 and EP-A-399,133), iminodiacetic acid-N-2-hydroxypropyl sulfonic acid and aspartic acid N-carboxymethyl N-2-hydroxypropyl-3-sulfonic acid (described in EP-A-516,102), β-alanine-N,N′-diacetic acid, aspartic acid-N,N′-diacetic acid, aspartic acid-N-monoacetic acid and iminodisuccinic acid chelants (described in EP-A-509,382), ethanoldiglycine acid, salts thereof and derivatives thereof.

EP-A-476,257 describes suitable amino based chelants. EP-A-510,331 describes suitable chelants derived from collagen, keratin or casein. EP-A-528,859 describes suitable alkyl iminodiacetic acid chelants. Dipicolinic acid and 2-phosphonobutane-1,2,4-tricarboxylic acid are also suitable.

Preferred aminocarboxylic chelants are diamine-N,N′-dipolyacid and monoamine monoamide-N,N′-dipolyacid chelants, salts thereof and derivatives thereof. Preferred polyacids contain at least two acid groups independently selected from the carboxylic acid group (—COOH), sulfonic group (—SO₃H), the o-hydroxyphenyl gorup, the m-hydroxyphenyl group and the p-hydroxyphenyl group. Suitable polyacids include diacids, triacids and tetraacids, preferably diacids. Preferred salts include alkali metal, alkaline earth, ammonium or substituted ammonium salts. EDTA is a particularly preferred chelant.

Preferably, the polyacids are di-carboxylic acids, preferably di-carboxylic acids having a carbon chain length of from about 3 to about 10 carbon atoms, more preferably from about 4 to about 6 carbon atoms, even more preferably about 4 carbon atoms.

Exemplary diamine dipolyacids suitable for use herein include ethylenediamine-N,N′-disuccinic acid (EDDS), ethylenediamine-N,N′-diglutaric acid (EDDG), 2-hydroxypropylenediamine-N,N′-disuccinic acid (HPDDS), all disclosed in European Patent EP 0 687 292, ethylenedicysteic acid (EDC) disclosed in U.S. Pat. No. 5,693,854, diaminoalkyldi(sulfosuccinic acids) (DDS) disclosed in U.S. Pat. No. 5,472,642 and EDDHA (ethylenediamine-N-N′-bis(ortho-hydroxyphenyl acetic acid)), a method of preparation of which is disclosed in EP 331,556. A preferred monoamine monoamide-N,N′-dipolyacid is glycinamide-N,N′-disuccinic acid (GADS), described in U.S. Pat. No. 4,983,315.

Highly preferred for use herein is ethylenediamine-N,N′-disuccinic acid (EDDS), derivatives and salts thereof. Preferred EDDS compounds for use hererin are the free acid form, and salts thereof. Preferred salts include alkali metal, alkaline earth metals, ammonium and substituted ammonium salts (e.g. monoethanolammonium, diethanolammonium, triethanolammonium). Highly preferred salts are sodium, potassium, magnesium and calcium salts. Examples of such preferred sodium salts of EDDS include Na₂EDDS and Na₃EDDS.

Preferred aminocarboxylic acid chelants that are not diamine-N,N′-dipolyacid and monoamine monoamide-N,N′-dipolyacid chelants include N,N′-bis(2-hydroxybenzyl)ethylenediamine-N,N′-diacetic acid (HBED) salts thereof and derivatives thereof.

### Polyphosphoric Acid Chelants

Suitable polyphosphoric acid type chelants include molecules that contain more than one P atom and have P—O—P bonds. Polyphosphoric acid chelants and salts (polyphosphates) can be linear and are generally represented by the formula [PₙO₃ₙ₊₁]⁽ⁿ⁺²⁾⁻ M₍ₙ₊₂₎⁺ wherein M is a suitable counter-ion such as H⁺, Na⁺ or K⁺ and n an integer. Polyphosphoric acid type chelants and their polyphosphate salts can also be cyclic and have the formula [PₙO₃ₙ]ⁿ⁻Mₙ⁺. Representative examples include, among other, sodium tripolyphosphate, tetrasodium diphosphates, hexametaphosphoric acid and sodium metaphosphate.

### Phosphonic Acid Chelants

Suitable phosphonic acid type chelants include amino alkylene poly (alkylene phosphonic acid), ethane 1-hydroxy diphosphonic acids and nitrilo trimethylene phosphonic acids, salts thereof and derivatives thereof. Suitable chelants of this type are disclosed in U.S. Pat. No. 4,138,478, Reese et al., U.S. Pat. No. 3,202,579 and U.S. Pat. No. 3,542,918, Berth et al., all incorporated herein by reference.

Preferred chelants for use herein are aminotri-(1-ethylphosphonic acid), ethylenediaminetetra-(1-ethylphosphonic acid), aminotri-(1-propylphosphonic acid), and aminotri-(isopropylphosphonic acid).

Especially preferred chelants are aminotri-(methylenephosphonic acid), ethylenediamine-tetra-(methylenephosphonic acid) (EDTMP) and diethylene-triaminepenta(methylenephosphonic acid) (DTPMP).

Amounts of the chelant will range from 10% to about 5%, preferably from 1.5 to 4%, optimally from about 2 to about 3% by weight of the colorant composition.

Part A compositions of this invention will include one or more ammonium carbonate or carbamate salts. These salts include ammonium carbonate, ammonium bicarbonate, ammonium carbamate and mixtures thereof. Further, these salts may include salt systems that through metathesis can form the aforementioned ammonium carbonate or carbamate salts. For instance, a combination of ammonium nitrate and sodium bicarbonate, ammonium phosphate and sodium carbonate, ammonium hydrogen phosphate and sodium carbamate may all exchange ions to form the ammonium carbonate or ammonium carbamate salts of this invention.

Amounts of the water soluble ammonium carbonate or carbamate may be present from about 0.01 to about 25%, preferably from about 0.1 to about 10%, optimally from about 1 to about 5% by weight of the colorant composition.

Certain embodiments of Part A of the present invention may in addition to ammonium carbonate or carbamate include ammonium hydroxide. Relative weight amounts of the ammonium carbonate or carbamate to the ammonium hydroxide may range from about 20:1 to about 1:1, preferably from about 10:1 to about 5:1.

Relative weight ratios of part A to part B may range from 5:1 to 1:5, preferably from 2:1 to 1:2, optimally about 1:1.

The dye composition according to the present invention may contain dye and coupler materials forming an oxidation dye precursor. Colors are produced by the reaction of the oxidation dye precursor with the oxidizing agent, such as hydrogen peroxide.

Suitable dyes include 1,4-diamino-benzene (p-phenylenediamine); 1,4-diamino-2-methyl-benzene (p-toluylenediamine); 1,4-diamino-2,6-dimethyl-benzene; 1,4-diamino-3,5-diethyl-benzene; 1,4-diamino-2,5-dimethyl-benzene; 1,4-diamino-2,3-dimethylbenzene; 2-chloro-1,4-diaminobenzene; 1,4-diamino-2-(thiophen-2-yl)benzene; 1,4-diamino-2-(thiophen-3-yl)benzene; 1,4-diamino-2-(pyridin-3-yl)benzene; 2,5-diaminobiphenyl; 1,4-diamino-2-methoxymethyl-benzene; 1,4-diamino-2-aminomethylbenzene; 1,4-diamino-2-hydroxymethyl-benzene; 1,4-diamino-2-(2-hydroxyethoxy)benzene; 2-(2-(acetylamino)ethoxy)-1,4-diaminobenzene; 4-phenylamino-aniline; 4-dimethylamino-aniline; 4-diethylamino-aniline; 4-dipropylamino-aniline; 4-[ethyl(2-hydroxyethyl)amino]-aniline; 4-[di(2-hydroxyethyl)amino]-aniline; 4-[di (2-hydroxyethyl)amino]-2-methyl-aniline; 4-[(2-methoxyethyl)amino]-aniline; 4-[(3-hydroxyropyl)amino]-aniline; 4-[(2,3-dihydroxypropyl)amino]-aniline; 1,4-diamino-2-(2-hydroxyethyl)-benzene; 1,4-diamino-2-(1-methylethyl)-benzene; 1,3-bis[(4-aminophenyl)(2-hydroxyethyl)amino]-2-propanol; 1,4-bis[(4-aminophenyl)amino]-butane; 1,8-bis(2,5-diaminophenoxy)-3,6-dioxaoctane; 4-aminophenol; 4-amino-3-methyl-phenol; 4-amino-3-(hydroxymethyl)-phenol; 4-amino-3-fluorophenol; 4-methylamino-phenol; 4-amino-2-(aminomethyl)-phenol; 4-amino-2-(hydroxymethyl)-phenol; 4-amino-2-fluorophenol; 4-amino-2-[(2-hydroxyethyl)-amino]methylphenol; 4-amino-2-methyl-phenol; 4-amino-2-(methoxymethyl)-phenol; 4-amino-2-(2-hydroxyethyl)-phenol; 5-amino-salicylic acid; 25-diamino-pyridine; 2,4,5,6-tetramino-pyrimidine; 2,5,6-diamino-4(1H)-pyrimidone; 4,5-diamino-1-1(2-hydroxyethyl)-1H-pyrazole; 4,5-diamino-1-(1-methylethyl)-1H-pyrazole; 4,5-diamino-1-[(4-methyl phenyl)methyl]-1H-pyrazole; 1-[(4-chlorophenyl)methyl]-4,5-diamino-1H-pyrazole; 4,5-diamino-1-methyl-1H-pyrazole; 2-aminophenol; 2-amino-6-methylphenol and 2-amino-5-methylphenol. The dyes may be used individually or together with each other in a mixture.

Suitable couplers for use with the dye include N-(3-dimethylamino-phenyl)-urea; 2,6-diamino-pyridine; 2-amino-4-[(2-hydroxyethyl)amino]anisole; 2,4-diamino-1-fluoro-5-methylbenzene; 2,4-diamino-1-methoxy-5-methylbenzene 2,4-diamino-1-ethoxy-5-methylbenzene; 2,4-diamino-1-(2-hydroxyethoxy)-5-methylbenzene; 2,4-di[(2-hydroxyethyl)amino]-1,5-dimethoxybenzene; 2,3-diamino-6-methoxy-pyridine; 3-amino-6-methoxy-2-(methylamino)pyridine; 2,6-diamino-3,5-dimethoxypyridine; 3,5-diamino-2,6-dimethoxy-pyridine; 1,3-diaminobenzene; 2,4-diamino-1-(2-hydroxyethoxy)benzene; 1,3-diamino-4-(2,3-hydroxpropoxy)benzene; 2,4-diamino-1,5-di (2-hydroxyethoxy)-benzene; 1-(2-aminoethoxy)-2,4-diaminobenzene; 2-amino-1-(2-hydroxyethoxy)-4-methylaminobenzene; 2,4-diaminophenoxyacetic acid ester; 3-[di(2-hydroxyethyl)amino]aniline; 4-amino-2-di [(2-hydroxyethyl)amino]-1-ethoxy-benzene; 5-methyl-2-(1-methyethyl)phenol; 3-[(2-hydroxyethyl)amino]aniline; 3-[(2-aminoethyl)amino]aniline; 1,3-di(2,4-diaminophenoxy)propane; di(2,4-diaminophenoxy)methane; 1,3-diamino-2,4-dimethoxybenzene; 2,6-bis(2-hydroxyethyl)aminotoluene; 4-hydroxyindole; 3-dimethylaminophenol; 3-diethylaminophenol; 5-amino-2-methylphenol; 5-amino-4-fluoro-2-methyl-phenol; 5-amino-4-methoxy-2-methyl phenol; 5-amino-4-ethoxy-2-methyl phenol; 3-amino-2,4-dichlorophenol; 5-amino-2,4-dichlorophenol; 3-amino-2-methyl-phenol; 3-amino-2-chloro-6-methyl phenol; 3-aminophenol; 2-[(3-hydroxyphenol)-amino]acetamide; 5-[(2-hydroxyethyl)amino]-4-methoxy-2-methyl phenol; 5-[(2-hydroxyethyl)amino]-2-methylphenol; 3-[(2-hydroxyethyl)amino]-phenol; 3-[(2-methoxyethyl)amino]-phenol; 5-amino-2-ethyl-phenol; 5-amino-2-methoxyphenol; 2-(4-amino-2-hydroxyphenoxy)ethanol; 5-[(3-hydroxypropyl)amino]-2-methyl phenol; 3-[(2,3-di hydroxypropyl)amino]-2-methylphenol; 3-[(2-hydroxyethyl)amino]-2-methylphenol; 2-amino-3-hydroxypyridine; 5-amino-4-chloro-2-methylphenol; 1-naphthol; 2-methyl-1-naphthol; 1,5-dihydroxy-naphthalene; 1,7-dihydroxy-naphthalene; 2,3-dihydroxynaphthalene; 2,7-dihydroxy-naphthalene; 2-methyl-1-naphthol-acetate; 1,3-dihydroxybenzene; 1-chloro-2,4-dihydroxy-benzene; 2-chloro-1,3-dihydroxybenzene; 1,2-dichloro-2,4-dihydroxy-4-methylbenzene; 1,5-dichloro-2,4-dihydroxy-benzene; 1,3-dihydroxy-2-ethyl-benzene; 3,4-methylenedioxy-phenol; 3,4-methylenedioxy-aniline; 6-bromo-1-hydroxy-3,4-methylenedioxybenzene; 3,4-diaminobenzoic acid; 3,4-di hydroxy-6-hydroxy-1,4(2H)benzoxazine; 6-amino-3,4-dihydro-1,4(2H)-benzoxazine; 3-methyl-1-phenyl-5-pyrazolone; 5,6-dihydroxyindole; 5,6-dihydroxyindolene; 5-hydroxyindole; 6-hydroxyindole; 7-hydroxyindole and 2,4-indolendione. The suitable couplers can be used individually, or together with each other in a mixture.

Self-coupling oxidation dye precursors include 2-amino-5-methylphenol; 2-amino-6-methyl phenol; 2-amino-5-ethoxyphenol and 2-propyl-amino-5-aminopyridine.

The dye composition may contain one or more alkalizing agents in addition to the ammonium carbonate or carbamate salts. These may be present in an amount from about 0.5 to 5% by weight of the colorant composition. The term “alkalizing agent” means an ingredient that is capable of imparting alkalinity (e.g. a pH of greater than 7) to the colorant formula. Suitable alkalizing agents include ammonium and metal hydroxides, alkanolamines, sodium silicates, sodium metasilicates, and mixtures thereof. Examples of metal hydroxides are sodium, potassium, lithium, calcium and magnesium hydroxide. A particularly preferred alkaline earth metal hydroxide is sodium hydroxide.

The colorant composition may contain one or more fatty acids, and if so suggested ranges are about 0.001-15%, preferably 0.005-10%, most preferably 0.01-8% by weight of the total composition. If fatty acids are present they will react with the alkalizing agent to form soap in situ, which provides a more shampoo-like character to the aqueous hair color composition once it is applied to hair. Suitable fatty acids include oleic acid, stearic acid, myristic acid, and linoleic acid. Particularly preferred is oleic acid.

The colorant composition may comprise one or more conditioners that exert a conditioning effect on hair. A variety of conditioners are suitable including cationic polymers, oily conditioning agents, silicones, fatty alcohols, proteins, and mixtures thereof. A combined total weight of conditioners may range from about 0.1 to about 25%, preferably 0.5 to 20%, more preferably 1 to 15% by weight of the colorant composition.

Illustrative cationic polymers are quaternary derivatives of cellulose ethers or guar derivatives, copolymers of vinylpyrrolidone, polymers of dimethyldiallyl ammonium chloride, acrylic or methacrylic polymers and quaternary ammonium polymers.

Examples of quaternary derivatives of cellulose ethers are polymers sold under the trademark JR-125, JR-400 and JR-30M. Suitable guar derivatives include guar hydroxypropyl trimonium chloride.

Oily conditioning agents are liquid at room temperature and may comprise esters and hydrocarbons. Examples include sunflowerseed oil, soybean oil, C₁₁₋₁₃ isoparafin and mineral oil. Amounts may range from about 0.001 to about 20%, preferably from 0.005 to 5%, more preferably from 0.01 to 10% by weight of the colorant composition.

Silicone hair conditioning agents include volatile or nonvolatile nonionic silicone fluids, silicone resins, and silicone semi-solids or solids.

Volatile silicones are linear or cyclic silicones having a measurable vapor pressure, which is defined as a vapor pressure of at least 2 mm at 20° C. Examples include cyclomethicones such as sold by Dow Corning under the DC 245, DC 244 and DC 344 designations.

Nonvolatile silicones may also be utilized. These can include polyalkyl siloxanes, polyaryl siloxanes, polyalkyl aryl siloxanes, polyether siloxane copolymers, amine-functional silicones and mixtures thereof.

Amounts of the silicone may range from about 0.001 to about 20%, preferably from about 0.005 to about 5, more preferably from about 0.01 to 4% by weight of the colorant composition.

The colorant composition may comprise one or more surfactants that assist in maintaining the emulsion form and aid in the foaming capability of the composition. Suitable surfactants include anionic, nonionic and amphoteric surfactants.

Amounts of the nonionic surfactant may range from about 0.01 to about ²⁰%, preferably from about 0.05 to 10%, more preferably from about 0.1 to 5% by weight of the total colorant composition. Suitable nonionic surfactants include alkoxylated alcohols or ethers, alkoxylated carboxylic acids, sorbitan derivatives. Examples include steareth-21, oleth-20, polysorbate and sorbitan oleate.

If desired the colorant composition may contain one or more anionic surfactants. Amounts may range about 0.1 to about 25%, preferably from 0.5 to 20%, more preferably from 1 to 15% by weight of the total combined composition. Suitable anionic surfactants include alkyl and alkyl ether sulfates generally having the formula ROSO₃M and RO(C₂H₄O)_{X}SO₃M wherein R is alkyl or alkenyl of from about 10 to 20 carbon atoms, x is 1 to about 10 and M is a water soluble cation such as ammonium, sodium, potassium, or triethanolamine cation.

It may be desirable to include one or more solvents in the colorant composition. Solvents assist in solubilizing the dye precursors in addition to other ingredients in the composition. The solvent when present may range from about 0.01 to 20%, preferably from 0.05 to 100%, more preferably from 0.1 to 6% by weight of the colorant composition. Suitable solvents include C₂₋₄ alkanols such as ethanol, isopropanol, and alkoxydiglycols such as ethoxydiglycol.

Relative weight ratios of the ammonium carbonate or carbamate salt to the chelant may range from about 10:1 to about 1:10, preferably from about 4:1 to about 1:2 optimally from about 2.5:1 to about 1:1 by weight.

Part B is a peroxide compound containing oxidizing composition sometimes referred to as a developer. Normally the peroxide compound will be hydrogen peroxide or a source which generates this material or a hydroperoxyl radical. Not only aqueous hydrogen peroxide but also precursors that can generate hydrogen peroxide may be employed. The precursors may include urea peroxide, sodium perborate, sodium percarbonate, potassium persulfate, calcium peroxide and sodium peroxide. The concentration of hydrogen peroxide may range from about 1% to about 50%, preferably from about 3% to about 30%, optimally from about 8% to about 20% by weight of the colorant composition.

A cosmetically acceptable carrier will be utilized with each of Parts A and B of the colorant composition. Suitable cosmetic carriers include water, alcohols, hydrocarbons and combinations thereof. Suitable alcohols include the C₁-C₄ lower alcohols and the C₂-C₄₀ polyols such as propylene glycol, polyethylene glycol, glycerine and combinations thereof. Hydrocarbons may include C₈-C₃₀ isoparaffins, C₃-C₇ hydrocarbons, polyalphaolefins and mixtures thereof. Water is the carrier of choice and normally will be the component present in largest amount both in Parts A and B. Amounts of the carrier may range from about 100% to about 99.9%, preferably from about 80% to about 99%, optimally from about 90% to about 95% by weight of the colorant composition.

Normally the oxidizing composition will have a low pH ranging from about 2 to about 6, preferably from 2.5 to 3.5. Buffering agents may often be employed within the oxidizing composition to maintain a desired pH level. Amounts of the buffering agents may from range from about 0.001 to about 2%, preferably from about 0.01 to about 0.1% by weight of the oxidizing composition. Phosphoric, hydrochloric, sulfonic and C₂-C₃₀ carboxylic acids and their salts are useful as buffering agents. Illustrative examples agents include tartaric acid, citric acid, acetic acid, lactic acid, ammonium sulfate, sodium dihydrogen phosphate/phosphoric acid, potassium chloride/hydrochloric acid, potassium dihydrogen phthalate/hydrochloric acid, sodium citrate/hydrochloric acid, potassium dihydrogen citrate/hydrochloric acid, sodium tartarate/tartaric acid, sodium lactate/lactic acid, sodium acetate/acetic acid, disodium hydrogen phosphate/citric acid and sodium chloride/glycine/hydrochloric acid and mixtures thereof. Most preferred is phosphoric acid.

The colorant compositions of the present invention can comprise a wide range of optional ingredients. Examples of these functional classes include: anticaking agents, antioxidants, binders, biological additives, bulking agents, chemical additives, cosmetic astringents, denaturants, drug astringents, emulsifiers, film formers, fragrance components, humectants, opacifying agents, plasticizers, preservatives, propellants, reducing agents, solvents, foam boosters, hydrotropes, solubilizing agents, suspending agents (nonsurfactant), sunscreen agents, ultraviolet light absorbers, and viscosity increasing agents (aqueous and nonaqueous).

It has been found by experimentation that daily hair care products can achieve durable desired hair color. As noted above, the daily hair care product consists of two parts.

Part A: dye intermediates at alkaline pH preferably in a conditioner or shampoo base

Part B: a peroxide such as hydrogen peroxide at acidic pH preferably in a conditioner or shampoo base

Part A is mixed with part B and the recently made mixture is applied to hair. The reason current hair coloring products come in two packages is because the mixture of the dye component and the oxidizing component is unstable and the two components must be kept apart until just before use. Similarly part A and part B of the present invention must be kept apart until just before use. By varying the concentration of the actives and the treatment time, the amount of color on hair may be varied while minimizing hair damage. To make the product more convenient and fool proof, part A and B may be packaged in dual dispensing systems where both parts are mixed outside of the package when dispensed and which is then applied to the wet hair as a conditioner or shampoo. Depending upon the amount of color desired, the treatment time could be varied from about 30 seconds to about 1 minute to about two minutes or longer.

Such conditioner or shampoo treatments would add color to hair gradually without damage due to lower contact time. Each subsequent treatment would add color until the desired shade is obtained. Depending upon the concentration of the actives and contact time, a desired shade may be reached in six to eight treatments. This process gives the user control over the amount of color deposited on the hair, and also the option to discontinue further applications if the color delivered is not to the user's liking. A user also has the option to switch to another color shade immediately without having to wait the six to eight weeks that is recommended for conventional treatments. With conventional hair color treatment, it is not recommended to perm and color hair simultaneously due to extensive damage. However, since this method colors the hair with minimum damage, perming can be done in the same time frame with this progressive coloring treatment.

The methods of the present invention are not as messy as conventional permanent hair coloring methods. The methods of the present invention do not use chemical compositions that are as smelly and noxious as those used in conventional permanent hair-coloring methods. Because the methods of the invention can be carried out in the shower, they do not involve the dripping and the mess associated with conventional permanent hair coloring. Parts A and B upon mixture form a composition with a pleasing viscosity and that is pleasing to the fingers. This effect may be enhanced where one or both of Parts A and B contain a cationic conditioning agent.

A dual package which can be employed in the products and kits of the present invention is disclosed in U.S. Pat. No. 6,082,588 to Markey et al which is hereby incorporated by reference.

Kit Containing an Instruction Sheet

The present invention also relates to a kit for carrying out the hair coloring method of the invention. The kit comprises a dye intermediate composition, an oxidizing composition and optionally a post treatment solution, each in a separate container or in a dual container, as described herein. The kit also contains written instructions that explain how the compositions of the invention are used.

The consumer admixes Parts A and B of the kit according to written instructions, to obtain a colorant composition mixture. The admixture may be conducted in a separate vessel external to the kit, or may take place in a container of the kit adapted to provide sufficient head space for mixing. Parts A and B may also be admixed on the hair of the user. Essentially upon mixing, reaction of the dye and the peroxide will commence. After treatment for a desired time the mixture is rinsed from the hair, preferably with water or a conventional shampoo or a conventional conditioning product.

As noted above, the mixture is applied to the hair and allowed to remain for a set time, usually about 30 seconds to about 1 to about 2 minutes to about 5 minutes or more.

In following the method of the invention, the consumer can compare the color of her hair with the desired hair color, which can be printed on the package of the product. The consumer can also vary the number of days of application of the product, and the consumer can also vary the amount of time the mixture of Parts A and B is left in the hair on each application. The number of applications can vary from 2 to about 30, preferably at least 6 treatments.

The method of the invention occurs over the course of days. Therefore, the final color of the consumer's hair may be affected by the amount of exposure to the sun of the hair during the course of treatment.

Desired hair color can also be reached by comparing hair after each treatment until it matches hair tresses taken from the consumer during a prior treatment.

Desired hair color can also be reached by testing the hair after each treatment with instruments, which measure the color of the hair. When the measurements of hair color of the treated hair reach a desired level, the treatment hair reach a desired level, the treatment can be stopped.

Indeed, reaching the desired hair color can be achieved by the use of any matching or comparison method commonly employed in the art.

Another aspect of the invention is that the ratio of the colorant composition (Parts A and B combined) relative to the consumer's hair may be applied in a weight ratio ranging from 1:10 to 1:2, preferably from 1:5 to 1:3, optimally about 1:4. Considerably more lifting of color with no additional damage is achieved by the aforementioned colorant composition to hair ratio, particularly where the ratio lies between 1:3 and 1:5. This is a distinct advantage and useful for any successful gradual daily hair colorant system.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

The following Example presents a two part composition for use with the kit and method of the present invention.

PART A

| Ingredient | Weight % |
|---|---|
| Stearamidopropyl Dimethylamine | 0.50 |
| Dicetyldimonium Chloride/PG (68:27) | 2.10 |
| Stearyl Alcohol and Ceteareth-20 (70% Active) | 1.00 |
| Cetyl Alcohol | 3.60 |
| Dimethicone | 1.00 |
| DC Silicone Fluid 245 | 1.80 |
| Sodium Metabisulfite | 0.15 |
| Erythobic Acid | 0.006 |
| 1,4-Aminophenol | 0.60 |
| 4-Amino-2-hydroxytoluene | 0.60 |
| Ammonium Carbonate | 7.40 |
| Ammonium Hydroxide (28% Active) | 3.60 |
| Sodium Hydroxide (50% Active) | 6.50 |
| Disodium Ethylenediamine Tetraacetic Acid | 1.90 |
| Deionized Water | To 100 |

*pH adjust to 9.8 with 50% sodium hydroxide.

Manufacturing Process:

1. Add deionized water to a flask and heat to about 85° C. under nitrogen, add EDTA and sodium metabisulfite followed by dyes.

2. Add Stearamidopropyl dimethylamine while stirring.

3. After all Stearamidopropyl dimethylamine has dissolved and the temperature in the flask is still at about 80° C. or above, add dicetyidimonium chloride/PG, dimethicone and DC silicone fluid 245.

4. Then add stearyl alcohol and cetereath-20 and then add cetyl alcohol.

5. When all the waxes are dispersed, increase agitator speed to moderate and mix at about 85° C. for about 10 minutes.

6. Let the batch cool to room temperature.

7. In a separate container prepare a solution of deionized water/NaOH at room temperature.

8. Into the solution dissolve ammonium carbonate optionally with ammonium salt.

9. Add this separate mixture into the main batch at room temperature and stir for 30 minutes.

10. Adjust the pH of the resulting mixture to between about 9.7 and 10.1.

Part B

| Ingredient | Weight % |
|---|---|
| Liquid Citric Acid (50%) | 0.20 |
| Stearamidopropyl Dimethylamine | 0.50 |
| Dicetyldimonium Chloride/PG (68:27) | 2.10 |
| Stearyl Alcohol and Ceteareth-20 (70%) | 1.00 |
| Cetyl Alcohol | 3.80 |
| Deionized Water | 4.10 |
| Disodium EDTA | 0.10 |
| Dimethicone | 1.00 |
| DC Silicone Fluid 245 | 1.80 |
| Hydrogen Peroxide (35%) | 10.00 |
| DMDM Hydantoin (55%) | 0.10 |
| Fragrance | 0.20 |
| Phosphoric Acid (85%) | 0.09 |
| Deionized Water | To 100 |

*pH = 2.5 to 3.5

Manufacturing Process:

1. Add item#1 into a suitable container. Heat to 85° C.

2. Add item#2 with moderate agitation.

3. Add item#4, dissolve completely.

4. At 75° C., add item#3, 5, 6

5. Mix batch at approximately 85° C. for at least an hour with agitation until particles are completely dissolved. Check for the particles.

6. Begin cooling.

7. In a separate container dissolve item#8 in item#7.

8. At 55° C. add the EDTA solution to the batch.

9. At 43° C. or below, add item# 9, 10, 11, 12, and 13 mix well.

10. Adjust the pH with item#14 to 2.7 to 3.0

11. Qs with water.

Method for Using Compositions of the Invention

Shampoo the hair as usual. Pump the composition of the invention into the palm and mix. Apply product to the hair like a regular conditioner. Rinse the hands. Leave product in the hair for example for about 30 seconds to about 5 minutes for color maintenance; and for example for about 5 minutes for color change. Rinse the hair thoroughly.

### EXPERIMENTAL

1. Formulation Stability: It is generally accepted that a marketed formulation for Hair Color needs to be stable for at least 3 months at 45° C. to survive market conditions globally. The formulations tested using carbonate salts as high as 10% in combination with CTAB can be shown to meet these criteria for stability.

2. Hair Tress Coloring: Caucasian hair tresses of the Piedmont type were supplied from IHIP. These tresses were about 6 to 9 inches in length and weigh about 9 grams. Hair Color measurements (L,a,b) were taken before and after treatment with a formulation using a Hunter Labscan XE. Each tress was first wet with water and treated with a formulation not containing dyes in a ratio of 1:1 for as short as 2 minutes and as long as 45 minutes. At least 3 tresses were used per formulation. Multiple treatments were also conducted to observe changes in lightening over time. These were generally on the order of 10, 20 and 30 consecutive treatments. All tresses were rinsed in water to remove any residual product and then measured for changes in lightening.

The results using a statistical design indicated that a Hair Coloring formula containing ammonium carbonate as low as 2% at pH 9.5 could be shown to lighten hair significantly better than a similar formula containing an equal molar weight of ammonia at pH 9.5. This was demonstrated for tresses treated singly for 45 minutes or after 10, 20 or 30 applications for 2 minutes each.

3. Hair Tress Bleaching: Caucasian hair tresses of either dark or medium brown were supplied from IHIP. The procedure and equipment was identical to that used to evaluate Coloring described above. Hunter measurements to evaluate the bleaching DE were calculated as the square root of (delta L)²+(delta a)²+(delta b)², where “delta” is the difference between initial and final color.

4. Trained Panel Assessment: Nine panelists trained in assessing damage of hair tresses were blinded in their evaluation of tresses treated with either ammonium carbonate or an ammonia containing Hair Coloring formulation. Tresses were treated 30 times for 2 minutes each with either of these formulations. The results indicated that the trained panelists ranked the ammonium carbonate tresses to be significantly less damaged than a similar formulation containing ammonia.

5. Color Enhancement: When Hair Coloring formulations contained oxidative dyes as PAP and PAOC, it could be shown that Piedmont hair treated with a formulation containing ammonium carbonate deposited more color than a similar formulation with ammonia as measured visually or using a Color instrument as the Hunter.

6. Mechanical Measurement of Tensile Properties: These studies employed standard instrument from Diastron—automated tensile tester model MTT 670. Prior to testing fiber diameters were assessed using commercially available Mitutoyo Laser Scan Micrometer. Single hair fiber tests were carried out on hair soaked in water for 30 min. During test, hair fiber (gauge length of 30 mm) was stretched at an extension rate of 20 mm/min during which load versus extension curves were recorded. The average of 60 hairs was used for each data point and four parameters of the tensile curves were compared: elastic modulus (slope of the linear portion of the curve, N/m²) break extension (extent of hair stretching before break, % of hair length extension) break stress (force needed to break fiber, N/m²) work to 20% extension (MJ/m²).

The results indicated that the changes in all tensile strength parameters on hair treated with a formulation containing ammonium carbonate are similar to the changes induced to the fiber by similar formulation containing ammonia. The carbonate treated hair can be shown being not more damaging than the formulation containing ammonia.

7. Combing force measurements were performed on an Instron 5542 tensile tester. A metal block which has a groove cut where the comb is placed, was secured to the base of tester with screws and was used as the comb holder in these combing force measurements. Each tress was manually combed three times (so that no tangles are left) before measurement. Root portion of hair tress was then mounted in a rigid clip secured to the movable cross-head of tester and free hanging portion of hair was pressed through to the base of the comb teeth. Position of the tress during combing was secured by the metal deflector inserted in front of the tress. 3.5 g tresses of 5 in length were used for evaluation. 2.5 g of product per 1 g hair was applied on hair for 5 min. After rinsing, dry and wet tress weight were compared and excess water squeezed out to minimize the variation in moisture content between tresses. Immediately afterwards measurement on wet tresses was performed. Dry combing is performed on tresses after a free-hanging drying for 24 hrs. Combing work (kgf) for each tress was calculated from the force (kg-mm) needed to comb through the tress over its entire length. Tests included one internal control—untreated hair tress wetted under the condition used for product application. From the results of these tests it could be seen that compositions of the invention which comprised ammonium carbonate had similar conditioning properties as similar compositions which did not have ammonium carbonate.

8. Cysteic Acid Test: Surface cysteic acid values were obtained on a Nicolet Spectrometer model Protégé 460 ESP, using a horizontal ZnSe flat plate from Thermo Spectra-Tech. In general, triplicate scans of each hair tress sample were acquired, each scan being the average of 32 scans. Surface cysteic acid was estimated (using an automated macro) as the ratio of the area of the cysteic acid peak (1001-1059 wave numbers) to the area of the amide III band (1196-1265 wave numbers). Cysteic acid is a marker for the oxidative damage to cysteine aminoacids in the hair fiber protein. The more damaging the treatment is to the hair the greater is the ratio of cysteic acid to amide III (fiber protein).

At the same time that hair damage is avoided by the methods of the invention, the consumer's hair is gradually being brought to the desired shade and color. This gradual change of color has three advantages: first, since the color is changed gradually, the consumer can stop the process if he or she does not like the color his or her hair is turning to. Second, some consumers do not want an abrupt change in color because they may feel embarrassed in public after having made such an abrupt change to the color of their hair. The methods of the present invention avoid such abrupt hair color changes. Third, the methods and compositions of the present invention can be used in the shower, and on a daily basis, because the compositions and methods of the present invention, by contrast with current color compositions, do not employ poisonous levels of chemicals and also because compositions and methods of the present invention involve hair application steps that are usually up to about two minutes to five minutes in length. By contrast current hair coloring compositions require approximately 30 minutes' time for each application, an amount of time, which is clearly not suitable for use in the shower.

### EXAMPLE 2

This Example focuses upon the interactive effect of ammonium carbonate and EDTA (a chelant typical of this invention). Experiments hereunder were conducted with part A and part B used in a 1:1 weight ratio as reported in the Tables below. Evaluations measuring Hair Damage and Bleaching excluded the dye components (p.aminophenol, N,N-bis(2-hydroxyethyl)-p-phenylenediamine Sulfate, p-phenylenediamine, resorcinol and m-phenylenediamine).

Part A

| Ingredient | Weight % |
|---|---|
| Citric Acid (50% Active) | 0.20 |
| Cetrimonium Chloride | 0.50 |
| Dicetyldimonium Chloride | 2.10 |
| Stearyl Alcohol and Ceteareth-20 | 1.00 |
| Cetyl Alcohol | 3.60 |
| Tetrasodium EDTA | * |
| Dimethicone | 0.10 |
| Cyclomethicone | 1.80 |
| p-aminophenol | 0.30 |
| N,N-bis(2-hydroxyethyl)-p- | 0.13 |
| phenylenediamine Sulfate | |
| p-phenylenediamine | 0.70 |
| Resorcinol | 0.45 |
| m-phenylenediamine | 0.04 |
| Ammonium Carbonate | * |
| Deionized Water | Balance |

* The asterisk represents values which were varied according to the experimental Table I-III below.

Part B

| Ingredient | Weight % |
|---|---|
| Liquid Citric Acid (50%) | 0.20 |
| Stearamidopropyl Dimethylamine | 0.50 |
| Dicetyldimonium Chloride/PG (68:27) | 2.10 |
| Stearyl Alcohol and Ceteareth-20 (70%) | 1.00 |
| Cetyl Alcohol | 3.80 |
| Disodium EDTA | 0.10 |
| Dimethicone (100%) | 1.00 |
| DC Silicone Fluid 245 | 1.80 |
| Hydrogen Peroxide (35%) | 10.00 |
| Phosphoric Acid (85%) | 0.09 |
| Deionized Water | Balance |

The Cysteic Acid Test (described above under Experimental procedure no. 8) was used to evaluate the amount of hair damage in the above systems using different levels of ammonium salt and chelant. Note that 4% ammonium carbonate is approximately equivalent to 0.75% ammonium hydroxide on a deliverable ammonium ion basis. The results are recorded in Table I below.

TABLE I
Ammonium Carbonate/EDTA Reduced Hair Damage

| Ammonium Carbonate/EDTA | Change In |
|---|---|
| (Weight %) | Cysteic/Amide II |
| 0.75% Ammonium Hydroxide/0% EDTA | 0.26 |
| 0.75% Ammonium Hydroxide/2% EDTA | 0.16 |
| 4% Ammonium Carbonate/0% EDTA | 0.18 |
| 4% Ammonium Carbonate/2% EDTA | 0.12 |

The results in Table I indicate that a combination of ammonium carbonate/EDTA was substantially more effective than ammonium carbonate alone in providing reduced hair damage.

Color deposition on hair was also enhanced through use of this invention. See the Experimental section above under procedure no. 2. Results are recorded in Table II below.

TABLE II
Ammonium Carbonate/EDTA Enhanced
Red Color Deposition On Hair

| Alkaline/Chelant (Weight %) | Change In “a” ** |
|---|---|
| 0.75% Ammonium Hydroxide/0% EDTA | 21.9 |
| 0.75% Ammonium Hydroxide/2% EDTA | 22 |
| 4% Ammonium Carbonate/0% EDTA | 24 |
| 4% Ammonium Carbonate/2% EDTA | 25.5 |

** Refers to redness factor “a” of Hunter Lab scale.

The results indicate that a combination of ammonium carbonate and tetrasodium EDTA enhanced red color deposition over ammonium carbonate alone, and was quite superior over ammonium hydroxide.

Enhancement of bleaching by a combination of ammonium carbonate/EDTA was evaluated in the tests reported under Table Ill. See the Experimental section above under procedure no. 3.

TABLE III
Ammonium Carbonate/EDTA Bleaching Effect

| Alkaline Agent/Chelant (Weight %) | Bleaching DE |
|---|---|
| 0.75% Ammonium Hydroxide/0% EDTA | 12 |
| 0.75% Ammonium Hydroxide/2% EDTA | 12.2 |
| 4% Ammonium Carbonate/0% EDTA | 20 |
| 4% Ammonium Carbonate/2% EDTA | 21 |

The results of the bleaching experiment indicate that there was no negative impact, and even some enhancement through addition of high levels EDTA to ammonium carbonate. The combination was especially effective compared to an equal molar amount of ammonium hydroxide.

### EXAMPLE 3

Another representative kit according to the present invention are the colorant and developer formulas part A and part B illustrated in the Tables below. The relative amounts used of part A and part B are in a weight ratio of 1:1. Application of the combined parts to hair are in a weight ratio of 1:4, respectively. These sets of ratios are to be referenced in all the subsequent Examples.

Part A

| Component | Weight % |
|---|---|
| Oleic Acid | 6.00 |
| C12-15 Pareth-9 | 1.00 |
| C12-15 Pareth-3 | 2.00 |
| PEG-2 Soyamine | 4.00 |
| Propylene Glycol | 7.00 |
| Isopropanol | 13.00 |
| Tetrasodium EDTA | 2.50 |
| Sodium Sulfite | 0.80 |
| Sodium Isoascorbate | 0.15 |
| P-Aminophenol | 0.75 |
| P-Amino-O-Cresol | 0.80 |
| Ammonium Carbamate | 6.00 |
| Water | To 100 |

Part B

| Component | Weight % |
|---|---|
| Aculyn 22 ® | 0.80 |
| Hydrogen Peroxide (50% Active) | 12.00 |
| Phosphoric Acid (85% Active) | 0.05 |
| Citric Acid (50% Active) | 0.10 |
| Water | To 100 |

### EXAMPLE 4

Another Example of the present invention are the colorant and developer formulas part A and part B outlined under the Tables.

Part A

| Component | Weight % |
|---|---|
| Sodium Lauryl Ether Sulfate (28% Active) | 3.00 |
| 2,5-Diaminotoluene Sulfate | 2.80 |
| Resorcinol | 1.00 |
| m-Aminophenol | 0.40 |
| 2-Amino-4-(2-Hydroxyethanolamino) Anisole Sulfate | 0.20 |
| Ascorbic Acid | 0.30 |
| Tetrasodium EDTA | 2.00 |
| Ammonia Carbonate | 4.20 |
| Ethanol | 2.00 |
| Water | To 100 |

PART B

| Component | | Weight % |
|---|---|---|
| Cetearyl Alcohol | | 10.00 |
| Aculyn 44 ® | | 4.00 |
| Hydrogen Peroxide (35% Active) | | 17.00 |
| Perfume | | 0.30 |
| Water | To | 100 |

### EXAMPLE 5

A still further illustration of formulas for a kit according to the present invention are the colorant and developer formulas part A and part B described below.

PART A

| Component | | Weight % |
|---|---|---|
| Cocoamidopropyl Betaine | | 5.00 |
| Ammonium Carbonate | | 2.20 |
| Sodium Citrate | | 0.40 |
| Stearalkonium Chloride | | 2.00 |
| p-Phenylenediamine | | 0.50 |
| Resorcinol | | 0.20 |
| Disodium EDDS | | 4.00 |
| Sodium Sulfite | | 0.10 |
| Perfume | | 0.10 |
| Water | To | 100 |

PART B

| Component | | Weight % |
|---|---|---|
| Hydrogen Peroxide | | 6.00 |
| Aculyn 33* | | 1.00 |
| EDIA | | 0.10 |
| Phosphoric Acid | | 0.10 |
| Water | To | 100 |

What is claimed is:

1. A method for permanently dyeing hair comprising subjecting a person's hair to a number of treatments, having a set time interval between each two consecutive treatments, wherein each treatment comprises:
a) contacting the hair with a recently prepared mixture of a colorant composition comprising:
A) an alkaline dye composition comprising:
(i) an effective amount to color hair of at least one dye intermediate;
(ii) from about 0.1 to about 25% by weight based on the colorant composition of a water soluble ammonium carbonate or carbamate salt;
(iii) from 1 to 5% by weight based on the colorant composition of a chelant; and
(iv) a cosmetically acceptable carrier;
B) an oxidizing composition comprising:
(i) from 0.1 to 15% by weight based on the colorant composition of a peroxide compound; and
(ii) a cosmetically acceptable carrier;
b) rinsing the mixture from the hair with water.

2. The method according to claim 1 wherein the chelant is selected from the group aminocarboxylic acids, amino phosphonic acids, polyphosphoric acids and mixtures thereof including their salts.

3. The method according to claim 1 wherein the chelant is a salt of ethylene diamine tetraacetic acid.

4. The method according to claim 1 wherein the ammonium salt is ammonium carbonate.

5. Th method according to claim 1 wherein the amount of ammonium carbonate or carbamate salt relative to the chelant is in a weight ratio of about 4:1 to about 1:2.

6. The method according to claim 1 wherein contact time on the hair is no longer than 2 minutes.

7. The method according to claim 1 wherein the number of treatments is at least six.

8. The method according to claim 1 wherein the colorant composition relative to an amount of a person's hair is applied in a weight ratio of about 1:10 to about 1:2.

9. A kit for permanently dyeing hair comprising:
a) a colorant composition packaged as a part A and a part B separate from one another until just before use,
part A being a dye intermediate composition comprising:
(i) an effective amount to color hair of at least one dye intermediate;
(ii) from about 0.1 to about 25% by weight of the colorant composition of a water soluble ammonium carbonate or carbamate salt;
(iii) from 1 to 5% by weight of the colorant composition of a chelant; and
(iv) a cosmetically acceptable carrier.
part B being an oxidizing composition comprising:
(i) from 0.1 to 15% by weight of the colorant composition of a peroxide compound; and
(ii) a cosmetically acceptable carrier.
b) instructions for use of the colorant composition comprising applying a recently made mixture of part A and part B onto the person's hair followed by a rinse with water.

10. The kit according to claim 9 wherein the chelant is selected from the group aminocarboxylic acids, amino phosphonic acids, polyphosphoric acids and mixtures thereof including their salts.

11. The kit according to claim 9 wherein the chelant is a salt of ethylene diamine tetraacetic acid.

12. The kit according to claim 9 wherein the ammonium salt is ammonium carbonate.

13. The kit according to claim 9 wherein the amount of ammonium carbonate or carbamate salt relative to the chelant is in a weight ratio of about 4:1 to about 1:2.

14. The kit according to claim 9 wherein the colorant composition relative to an amount of a person's hair is applied in a weight ratio of about 1:10 to about 1:2.
